(19) United States
(12) Patent Application Publication
(10) Pub. No.: US 2016/0235872 A1
(43) Pub. Date: Aug. 18, 2016

(54) Compositions and Methods to Image and Quantify Inflammation

(71) Applicant: Celsense, Inc.

(72) Inventors: Brooke M. Helfer, Houston, PA (US); Anthony G. Balducci, Pittsburgh, PA (US); Charles F. O'Hanlon, III, Pittsburgh, PA (US)

(73) Assignee: Celsense, Inc., Pittsburgh, PA (US)

(21) Appl. No.: 15/043,004

(22) Filed: Feb. 12, 2016

Related U.S. Application Data
(63) Continuation of application No. 14/783,684.

(51) Int. Cl. A61K 51/12; A61B 5/055; A61K 51/04

(52) U.S. Cl. CPC A61K 51/1203; A61K 51/0423; A61B 5/055

(57) ABSTRACT

Compositions and methods for assessing inflammation in a subject. The preset disclosure provides compositions for labeling leukocytes with a ¹⁹F-containing perfluoropolyether molecule ex vivo. In some examples, the leukocytes are obtained from the patient, enriched in a whole blood fraction, and then labeled. The labeled cells may be re-introduced into the patient. The leukocytes may accumulate at a site of inflammation, thus permitting non-invasive evaluation of inflammation in patients. The present methods provide a tool for assessing inflammation in a wide variety of autoimmune diseases and may have particular utility in intestinal diseases such as Crohn's disease, ulcerative colitis, inflammatory bowel disease, and cardio myositis.

### CROSS-REFERENCE TO RELATED APPLICATIONS

[1] This application is a continuation of U.S. application Ser. No. 14/783,684 filed Oct. 9, 2015, which in turn is a national stage entry of PCT Application No. PCT/US14/52685 filed Aug. 26, 2014, which claims the benefit under 35 U.S.C. §119(e) of the earlier filing date of U.S. Provisional Patent Application No. 61/912,832 filed on Dec. 6, 2013 and, U.S. Provisional Patent Application No. 61/920,498 filed on Dec. 24, 2013.

### BACKGROUND OF THE INVENTION

1. Field of the Invention

The present invention relates generally to compositions and techniques for assessing inflammation. More particularly, the present invention is directed to compositions useful in conducting magnetic resonance imaging (MRI)-based assessment of inflammation.

2. Description of the Background

Many biological processes are carried out by populations of cells. For example, cells of the immune system are recruited from the body to areas of injury or infection, resulting in an accumulation of leukocytes at the affected site. A marked infiltration of leukocytes often occurs in tissues affected by autoimmune diseases, physical injury, cancers, organ transplantation rejection, and infections.

Although migration of leukocytes plays a key role in numerous diseases, present technologies available for noninvasively monitoring the location and quantity of leukocytes in vivo are limited. Typically, detecting the accumulation of leukocytes is accomplished by a histological analysis of biopsied tissues. The tissue sampling process is invasive and a large number of samples may be needed to ascertain the macroscopic location and extent of cellular infiltration. Repetitive sampling of an inflamed tissue may result in further inflammation of the tissue, confounding results obtained by the process.

Autoimmune diseases of the intestines are prevalent in humans. Studies have shown that diseases such as Crohn's, ulcerative colitis, other embodiments of inflammatory bowel disease (IBD) and certain intestinal cancers are accompanied by an accumulation of leukocytes in the intestinal tissue. Studies have also shown that the location and quantity of leukocytes in the tissue is heterogeneously distributed in both the subject and the population. Detecting the location and quantity of leukocytes in the intestinal tissue prior to selecting a treatment option may lead to better outcomes for these diseases.

Cardio myositis is also an autoimmune disease that is prevalent in humans. This disease causes a swelling of the heart muscle, and typically presents with few symptoms beyond generalized fatigue and a shortness of breath. If untreated, the disease may cause lasting or permanent damage to the heart. In this disease, the cause of the inflammation may be an infection, injury, or the unwanted side effect of a medical treatment. In many cases, the cause of the disease is unknown, or idiopathic. Detecting the quantity of location and leukocytes in the heart in patients may lead to better outcomes for this disease.

According to the World Health Organization, over 100,000 human organs are transplanted each year. In the US alone, over 25,000 human organs are transplanted each year. A healthy human immune system recognizes the foreign organ as a pathogen, and attempt to kill the pathogen by surround the organ with leukocytes. This response is called organ transplant rejection. In all cases, the patient must be treated with anti-rejection drugs to down regulate the patient's immune system to prevent permanent damage to the new organ. To assure proper dosage of the anti-rejection drug, tissue samples from the foreign organ are harvest on a regular basis and inspected for leukocytes. This is a dangerous procedure and subject to error due an observed inhomogeneity of leukocytes on the surface of the organ.

Existing instruments for non-invasive analysis of the trafficking of leukocytes are ill-suited for clinical use. Light-based imaging technologies, such as bioluminescence (e.g., luciferases) technologies, are often ineffective at visualizing deep structures because most mammalian tissues are optically opaque. Radioactive probes used in Positron Emission Tomography (PET) and/or Single Photon Emission Computed Tomography (SPECT) are highly sensitive, but the spatial resolution of cellular location is limited. These techniques must be combined with either CT or MRI to place the detected leukocytes into anatomical context, requiring more complicated analysis and/or instrumentation. Furthermore, longitudinal analysis of leukocyte location is limited by the time scale of the radioactive decay of the probe.

Magnetic resonance imaging (MRI) is a widely used clinical diagnostic tool because it is non-invasive, allows views into optically opaque subjects, and provides contrast among soft tissues at reasonably high spatial resolution. Conventional MRI focuses almost exclusively on visualizing anatomy and has no specificity for any particular cell type. Fluorine (¹⁹F) MRI employs the same physical phenomena as conventional (proton, ¹H) MRI, but probes frequencies unique to the fluorine atom. Since fluorine is present, if at all, at exceedingly low levels in living organisms, ¹⁹F MR images do not contain endogenous background signal and are highly specific to exogenous contrast agents. Further, with appropriate operation of the MRI instrument, the signal in a single volume element (voxel) acquired in ¹⁹F MRI is directly proportional to the number of fluorine atoms present in the location represented by the voxel.

There has been a long-standing need in the medical community for non-invasive tools for reliably assessing inflammation in patients. The present invention addresses this need.

### SUMMARY OF THE INVENTION

The present invention addresses the limitations currently existing within the art and provides a quantitative tool to assess the degree of and location of inflammation in a disease state through the detection of accumulating lymphocytes.

The present invention provides emulsions useful for labeling cellular populations such as leukocytes. The emulsions may contain a ¹⁹F-containing molecule, a surfactant, and water, with an average particle size between about 10 nanometers and 500 nanometers. In some embodiments, the average particle size is between about 135 nanometers and 195 nanometers. The ¹⁹F-containing molecule may be present at about 5% by weight and about 15% by weight of the emulsion. The surfuctant may be present from about 0.1% to about 1% by weight of the emulsion. In some embodiments, the surfactant is a non-ionic surfactant and in some instances, the non-ionic surfactant is the commercially available PLURONIC 68. The ¹⁹F-containing molecule may be a linear perfluoropolyether or a cyclic perfluoropolyether. A particularly useful cyclic perfluoropolyether is perfluoro-15-crown-5-ether. A particularly useful set of linear perfluoropolyethers have a formula of CF₃O(CF₂CF₂O)ₙCF₃ where n is between 8 and 14.

The present invention also provides a method of labeling leukocytes. The method includes the steps of obtaining leukocytes from a patient, forming a cellular suspension of the leukocytes in a physiological medium. The leukocytes may be used as obtained from the patient (i.e., whole blood), enriched in a medium (e.g., the buffy coat resulting from a centrifugation product), or isolated from other cell types. The cellular suspension may be combined with an amount of an emulsion of a ¹⁹F-containing molecule, a surfactant, and water, as described above. The cellular suspension plus emulsion may be incubated for a sufficient amount of time to allow the ¹⁹F-containing molecule to associate with the leukocytes. In some embodiments, about 0.25 mg to about 20 mg of emulsion per milliliter of cellular suspension is added to the cellular suspension. Following incubation the leukocytes may be associated with the ¹⁹F-containing molecular label.

After labeling, the leukocytes may be reintroduced into the patient's body. In some particularly useful embodiments, the leukocytes are injected directly into the circulatory system of the patient. The labeled leukocytes may then be allowed to travel throughout the body and react to physiological stimuli normally. When a medical practitioner wishes to learn about the location of the leukocytes, the patient may be scanned using conventional magnetic resonance imaging (MRI) techniques. A conventional structural ¹H MRI may be conducted to obtain an anatomical image of the patient. The patient may then be examined using ¹⁹F MRI, which (given the unique magnetic signature of ¹⁹F) will reveal the location of the labeled leukocytes. By overlapping the ¹H and ¹⁹F MRI data sets, the medical practitioner may identify the location and anatomical structure where labeled leukocytes have collected in the body of the patient.

The compositions and methods of the present invention are useful in a wide variety of medical contexts as a way of evaluating inflammation in a subject. Leukocytes commonly are enriched in areas of inflammation. Therefore, the compositions and methods of present invention permit assessment of the areas and severity of inflammation in a patient non-invasively. The site of inflammation may be the intestines, the heart, a transplanted organ, an endocrine-secreting organ, the central nervous system, a cancer or tumor, or a site of localized infection of the subject. The methods and compositions of the present invention are particularly useful for assessment of inflammation Crohn's, IBD, ulcerative colitis, cardio myositis, and organ transplantation, among other medical situations and conditions.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 is a schematic of a method of the present invention;

FIG. 2A depicts the degree of labeling of various cell types for a four hour incubation using the methods of the present invention; and

FIG. 2B depicts the degree of labeling of various cell types for a 24 hour incubation using the methods of the present invention.

### DETAILED DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known. The detailed description will be provided herein below with reference to the attached drawings.

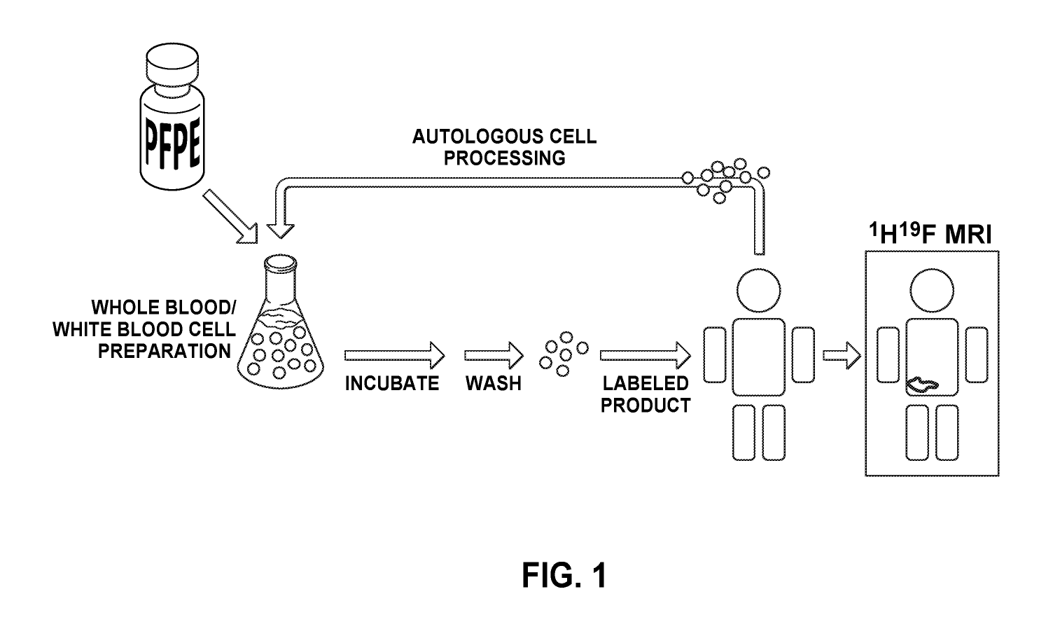

This disclosure discloses a novel method of non-invasively assessing inflammation in a patient. A schematic of the general methods of the present invention is depicted in FIG. 1. As part of the methods of the present invention, a portion of a subject's leukocytes are removed from the subject, labelled with an agent detectable in ¹⁹F MRI, and re-injected into the subject. After some time, the subject, or some portion thereof, is interrogated using ¹⁹F MRI where the labelled cells are rendered distinct from the subject. The labelled cells serve as a proxy measure of the trafficking of leukocytes in a subject. In certain aspects, a method of the invention may comprise acquiring a conventional ¹H image and the ¹⁹F image during the same imaging session, without moving the subject. Overlay of the ¹⁹F and ¹H images provides information concerning location in the anatomy and the intensity of the ¹⁹F image provides information concerning leukocyte concentrations.

The methods of the present invention include the step of extracting cells from a patient and labeling those cells with a ¹⁹F-containing reagent. The extracted cells may be immediately labeled without further processing. Alternatively, once extracted, the cellular suspension may be enriched in a particular cell type (i.e., the percentage of cells to be labeled in the composition is increased) or may be processed so that a desired cell type is isolated (i.e., the cells to be labeled are specifically separated from other, non-desired cells).

Within the context of the present invention, any method of isolating a particular cell type or enriching a composition with a particular cell type (or multiple cell types) may be used. Such methods range from basic centrifugation techniques to more sophisticated immunologically based processes. One of skill in the art will be familiar with the range of processes available for isolation or enrichment of particular cell types.

In certain embodiments, the cells to be labeled are a component of blood. Specifically, cells that are associated with the expression of inflammation (e.g., leukocytes) are particularly useful within the context of the present invention. Leukocytes are primarily white blood cells that include macrophages, T- and B-lymphocytes, neutrophils, eosinophils, and other less common cell types. In some particularly useful embodiments of the present invention, leukocytes or sub-populations of leukocytes may be associated with ¹⁹F-containing label to generate a tool useful for assessing inflammation in a subject.

Leukocytes or sub-populations of leukocytes may be obtained either by simply using whole blood extracted from a patient or by enriching or isolating leukocytes in a composition. While sophisticated isolation processes may be used, a simple centrifugation-based technique may also be employed to generate a cellular composition for labeling of leukocytes. For example, blood may be drawn from a patient using traditional phlebotomy techniques. The whole blood may be subjected to density gradient centrifugation, causing fractionation of the blood. After centrifugation, the blood separates into three main layers—a clear layer of plasma at the top, a red portion containing mostly red blood cells at the bottom, and a thin layer in between those two. The thin layer is commonly referred to as the buffy coat and contains most of the white blood cells and platelets. The buffy coat may be used within the context of the present invention as an easily obtained composition that is enriched in leukocytes.

Once the buffy coat is isolated, the cells may be resuspended in physiological solutions appropriate for labeling of the cells. One of skill in the art will recognize numerous appropriate physiological media (e.g., Ringer's, Hartmann's) that may be used to resuspend cells. Prior art methods of labeling leukocytes have included cationic lipids and uptake-promoting reagents. While these methods are effective, some of the reagents cause a degree of cell death. Of course, this limits the physiological utility of the labeled cells. It has been found that, within the context of the present invention, a simplified composition may be used for labeling leukocytes. Specifically, compositions that include low levels of a surfactant and a ¹⁹F-containing label emulsified in water for injection provide both superior cell viability as well as substantial labeling of leukocytes. Through this exposure, the leukocytes become labeled through association with the ¹⁹F-containing compounds.

Within the context of the present invention, it has been found that perfluoropolyethers (PFPEs) are particularly useful in ex vivo labeling of leukocytes. PFPEs may be linear or cyclic molecules and include perfluoro crown ethers. Examples of PFPE molecules that may be utilized within the context of the present invention are shown in U.S. Pat. No. 8,449,866, which is hereby incorporated by reference. In certain embodiments, the ¹⁹F-containing imaging reagent is a perfluoro crown ether, such as perfluoro-15-crown-5, perfluoro-18-crown-6, perfluoro-2-crown-4, etc., also referred to as cyclic perfluoropolyethers (cyclic PFPEs). Such compounds are advantageous in that the ¹⁹F nuclei of these molecules will have similar or identical NMR resonances, resulting in a higher signal-to-noise ratio image with a reduction in or absence of chemical-shift image artifacts. The macrocycle perfluoro-15-crown-5-ether has particularly preferable characteristics as it provides a strong signal, while at the same time stably labeling leukocytes. Esters of perfluoro-tert-butanol, 1,3,5-tris(trifluoromethyl)benzene, hexafluoroacetone, poly(trifluoromethylethylene), and perfluorocyclohexane are examples of compounds having multiple fluorine atoms with ¹⁹F resonances that have the same, or nearly the same, Larmor frequencies.

Similarly, the ¹⁹F-containing imaging reagent may also be a polymer. In certain embodiments, the imaging reagent is or includes a linear PFPE, e.g., a compound having a structure or portion thereof comprising repeated units of —[O—CF₂(CF₂)ₓCF₂]ₙ—, where x is an integer from 0 to 10, preferably from 0-3, and n is an integer from 2 to 100, preferably from 4 to 40. It has been found that linear PFPE having the formula CF₃O(CF₂CF₂O)ₙCF₃, where n is between 8 and 14 is particularly useful within the context of the present invention.

The compositions for labeling cells may also include a surfactant. It has been found that surfactants may be used advantageously within the context of the present invention to label cells. The surfactant may be cationic, anionic, neutral, and amphoteric in nature. In some embodiments, a non-ionic surfactant has been found to be particularly useful. One of skill in the art will be aware of numerous non-ionic surfactants that may be utilized within the context of the present invention, including polyoxyethylene glycol alkyl ethers, polyoxypropylene glycol alkyl ethers, glucoside alkyl etherse, polyoxyethylene glycol octylphenol ethers, sorbitan alkyl esters, and glycerol alkyl esters. Particular embodiments of the present invention employ the commercially available non-ionic surfactant PLURONIC 68.

Within the context of the present invention, a composition may be generated using the components listed above in the following manner. In certain embodiments, the surfactant may be included from about 0.1 to about 1% by weight of the solution, and the PFPE may be present from about 5% to about 15% by weight of the composition.

In certain embodiments of the present invention, the PFPE-containing composition is emulsified prior to exposure to the cells to be labeled. In certain embodiments, the emulsion is stable at room temperature and at body temperature. It has been found that emulsions having an average particle size of between about 10 and about 500 nm in diameter are useful within the context of the present invention. Emulsions having an average particle size of between about 135 and 195 nm in diameter have been found to be particularly useful within the context of the present invention.

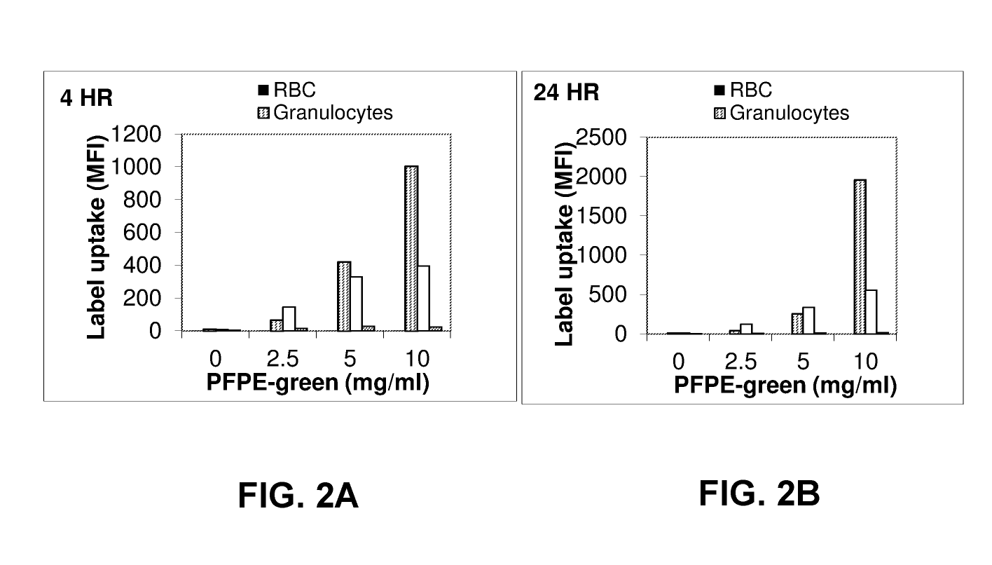

For labeling cells, an aliquot of the PFPE-containing emulsion is placed into the cellular suspension. The specific amount of emulsion added to the cellular suspension may vary and will depend on the density of cells in the suspension and PFPE utilized. In general, about 0.25 to about 20 milligrams of the emulsion is added for every milliliter of cellular suspension to generate a cell labeling solution. The leukocytes may be incubated in this labeling solution for between about 2 hours to about 24 hours. One of skill in the art may select the duration of incubation based on the appropriate level of association between the PFPE and the cells. Using a cellular suspension of whole blood, leukocytes may be effectively labeled as shown in FIG. 2. FIG. 2A displays the labeling of various cell types found in whole blood following an incubation of four hours with the labeling solution, as described above. FIG. 2B shows the labeling of various cell types found in whole blood following an incubation of 24 hours in the labeling solution, as described above.

Following exposure to the labeling solutions, the leukocytes may be washed to remove excess PFPE. The cells may then be resuspended in solutions appropriate for reinjection into the patient. Once reinjected into the patient, the leukocytes are able to function and respond to immunological stimuli normally.

The present invention takes advantage of the medically observed fact that leukocytes often accumulate at sites of inflammation in the body. The labeled leukocytes represent a small proportion of the overall leukocyte population in the patient's body, but at the same time they provide a unique signature when assessed using MRI techniques. As such, accumulation of a ¹⁹F signal at a particular location in a tissue reflects a measure of the inflammation of that portion of tissue. To identify the anatomical structure that is labeled by the ¹⁹F signal, it may be appropriate to conduct a structural ¹H MRI scan of the area of the patient's body. Overlay of the ¹H and ¹⁹F images provides a medical practitioner with valuable information about the location and degree of inflammation in the patient.

The methods of the present invention may be used to evaluate inflammation in a numerous disease states. For example, gastrointestinal, cardiovascular, neurological, endocrinological, pulmonary, and muscular-skeletal diseases that include inflammation of specific tissues may be evaluated. The methods of the present invention may be used to assess inflammation in multiple locations within the subject, including the intestines, the heart, a transplanted organ, an endocrine-secreting organ, the central nervous system, a tumor in the subject, or a site of localized infection in the subject. As a specific example, the gastrointestinal diseases of Crohn's disease, ulcerative colitis, and inflammatory bowel disease often present with an accumulation of leukocytes at the intestinal site of inflammation. The location of the inflammation (e.g., distal or proximal intestines for Crohn's disease) may be significant marker for the prognosis or treatment strategy for the disease state. This information may be readily obtained using the methods and compositions of the present invention. Additionally, due to the stable nature of ¹⁹F, the present invention permits longitudinal studies of leukocyte accumulation previously unavailable in the prior art. The duration of the longitudinal studies will be primarily constrained by the cellular turnover rate that is specific to the cells

The present invention provides a skilled practitioner with a substantial tool to evaluate inflammation non-invasively. The medical practitioner may avoid inflicting the patient with painful biopsy procedures that are of limited interpretive value.

Nothing in the above description is meant to limit the present invention to any specific materials, geometry, or orientation of elements. Many part/orientation substitutions are contemplated within the scope of the present invention and will be apparent to those skilled in the art. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered only to facilitate comprehension of the invention and should not be construed to limit the scope thereof.

What is claimed is:

1. An emulsion useful for labeling leukocytes, comprising:
a ¹⁹F-containing molecule;
a surfactant; and
water, wherein the emulsion has an average particle size between about 10 nanometers and about 500 nanometers.

2. The emulsion of claim 1, wherein the ¹⁹F-containing molecule is present from about 0.5% to about 15% by weight of the emulsion.

3. The emulsion of claim 1, wherein the surfactant is present from about 0.1 to about 1% by weight of the emulsion.

4. The emulsion of claim 1, wherein the ¹⁹F-containing molecule is a linear perfluoropolyether or a cyclic perfluoropolyether.

5. The emulsion of claim 5, wherein the linear perfluoropolyether has a formula of CF₃O(CF₂CF₂O)ₙCF₃ where n is between 8 and 14.

6. The emulsion of claim 5, wherein the cyclic perfluoropolyether is perfluoro-15-crown-5-ether.

7. The emulsion of claim 1, wherein the surfactant is a non-ionic surfactant.

8. The emulsion of claim 1, wherein the non-ionic surfactant is PLURONIC 68.

9. The emulsion of claim 1, wherein the emulsion has an average particle size between about 135 and about 195 nanometers.

10. A method of labeling leukocytes, comprising:
obtaining leukocytes from a patient;
forming a cellular suspension comprising the leukocytes;
combining the cellular suspension with an amount of an emulsion of ¹⁹F-containing molecule, a surfactant, and water, where the emulsion has an average particle size of between about 10 nanometers and about 500 nanometers to form a cell labeling composition; and
incubating the leukocytes in the cell labeling composition.

11. The method of claim 10, wherein the amount of emulsion is about 0.25 mg to about 20 mg of emulsion per milliliter of cellular suspension.

12. The method of claim 10, wherein the ¹⁹F-containing molecule is a linear perfluoropolyether or a cyclic perfluoropolyether.

13. The method of claim 12, wherein the linear perfluoropolyether has a formula of CF₃O(CF₂CF₂O)ₙCF₃ where n is between 8 and 14.

14. The method of claim 12, wherein the cyclic perfluoropolyether is perfluoro-15-crown-5-ether.

15. The method of claim 10, wherein the average particle size is between about 135 nanometers and about 195 nanometers.

16. A method of assessing inflammation in a subject, comprising the steps of:
obtaining leukocytes from the subject;
labeling the leukocytes by incubating the leukocytes with an emulsion of ¹⁹F-containing molecule, a surfactant, and water, where the emulsion has an average particle size of between about 10 nanometers and about 500 nanometers;
introducing the labeled leukocytes into the subject;
allowing the labeled leukocytes to travel throughout a body of the subject;
examining the subject using conventional ¹H MRI to obtain a ¹H MRI data set;
examining the subject using ¹⁹F MRI to obtain a ¹⁹F MRI data set; and
overlaying the ¹⁹F MRI data set and the ¹H MRI data set to identify the location of the labeled leukocytes within the subject.

17. The method of claim 16, wherein the location of the labeled leukocytes is a site of inflammation in the subject.

18. The method of claim 16, wherein an image intensity associated with the ¹⁹F MRI data set is used to measure a number of leukocytes at the site of inflammation in the subject.

19. The method of claim 16, wherein the location and image intensity are used to assess the severity of inflammation at the site of inflammation.

20. The method of claim 16, wherein the site of inflammation is the intestines of the subject, the heart of the subject, a transplanted organ in the subject, an endocrine-secreting organ of the subject, the central nervous system of the subject, a cancer of the subject, or a site of localized infection of the subject.
